(19) United States
(12) Patent Application Publication
(10) Pub. No.: US 2025/0177768 A1
(43) Pub. Date: Jun. 5, 2025

(54) SENSING AND STIMULATION DEVICE

(71) Applicant: NATIONAL TSING HUA UNIVERSITY, Hsinchu City (TW)

(72) Inventor: Ting-Wei WANG, Hsinchu City (TW)

(73) Assignee: NATIONAL TSING HUA UNIVERSITY, Hsinchu City (TW)

(21) Appl. No.: 18/606,362

(22) Filed: Mar. 15, 2024

(30) Foreign Application Priority Data
Dec. 5, 2023 (TW) 112147170

(51) Int. Cl. A61N 2/00; A61N 2/02

(52) U.S. Cl. CPC A61N 2/006; A61N 2/02

(57) ABSTRACT

A sensing and stimulation device arranged on an ear of a subject is disclosed. The device includes a coil structure, a setting structure combined with the coil structure, and a control module coupled to the coil structure. The setting structure is configured to position the coil structure on the ear. The control module is configured to drive the coil structure for an eddy current induction measurement to an ear measurement area, and to drive the coil structure for an electromagnetic stimulation to an ear stimulation area. The control module controls at least one parameter of the electromagnetic stimulation based on at least one measurement result of the eddy current induction measurement.

### TECHNICAL FIELD

The present invention relates to a sensing and stimulation device. Specifically, the present invention relates to an ear-worn eddy current sensing and electromagnetic stimulation device.

### BACKGROUND

In today's high-pressure society that emphasizes speed and competition, autonomic nervous system disorder has become one of the common problems among modern people. The autonomic nervous system (sympathetic and parasympathetic nerves) is the fully automatic control center of the human body. Functions such as heartbeat, blood pressure, respiration, digestive tract, bladder, and gland secretion are all regulated by the autonomic nervous system. Autonomic nervous disorders are mostly caused by the sympathetic nerve being overly excited by external stimulation, which causes the patient to have negative reactions such as panic, anxiety, and accelerated heartbeat, thus affecting the patient's health.

For the monitoring and regulation of autonomic nerves, there are currently many wearable devices used to monitor or stimulate autonomic nerves. However, in prior art, wearable devices were only designed for a single function such as monitoring or stimulation. Moreover, in prior art, sensing or stimulating autonomic nerves requires electrodes and gel as a medium between the skin and the electrodes. Therefore, if long-term autonomic nervous system monitoring or regulation is required, the gel and/or electrodes will be in contact with the skin for a long time, which may cause discomfort such as skin allergies in the subject. In addition, wearing electrodes can easily lead to problems such as falling off or electrode displacement due to movement, sweating, or accidental touching by the subject.

From the above, it can be seen that there are still many problems that need to be overcome in prior art when it comes to wearable autonomic nervous system monitoring and control devices.

### SUMMARY

Therefore, the present invention proposes a sensing and stimulation device arranged on an ear of a subject, in order to effectively solve the problems encountered in prior art.

One objective of the present invention is to provide an integrated sensing and stimulation device that allows sensed values to be fed back to the device and to adjust stimulation parameters.

Another objective of the present invention is to provide a sensing and stimulation device that is easy to wear for a long time and is not easily affected by the wearer's actions.

Yet another objective of the present invention is to provide a sensing and stimulation device that avoids the use of contact electrodes.

One embodiment according to the present invention is directed to a sensing and stimulation device arranged on an ear of a subject. The sensing and stimulation device includes a coil structure, a setting structure combined with the coil structure, and a control module coupled to coil structure. The setting structure is configured to fix the coil structure on the ear. The control module is configured to drive the coil structure for an eddy current induction measurement to an ear measurement area, and to drive the coil structure for an electromagnetic stimulation to an ear stimulation area. The control module controls at least one parameter of the electromagnetic stimulation based on at least one measurement result of the eddy current induction measurement.

In a preferred embodiment, the coil structure includes a first coil unit arranged corresponding to the ear measurement area, and a second coil unit arranged corresponding to the ear stimulation area.

In a preferred embodiment, the coil structure includes a substrate and at least one coil arranged on the substrate.

In a preferred embodiment, the ear measurement area corresponds to a vein location of the ear.

In a preferred embodiment, the ear stimulation area corresponds to a parasympathetic nerve location of the ear.

In a preferred embodiment, the at least one measurement result of the eddy current induction measurement includes a heart rate signal of the subject. The heart rate signal includes low-frequency part and a high-frequency part, and the control module controls the at least one parameter of the electromagnetic stimulation based on a ratio of the low-frequency part to the high-frequency part.

In a preferred embodiment, the control module drives the coil structure for the electromagnetic stimulation when the ratio is greater than a threshold.

In a preferred embodiment, the control module drives the coil structure for the eddy current induction measurement after the electromagnetic stimulation, obtains an updated heart rate signal, and determines whether to repeat the electromagnetic stimulation based on the updated heart rate signal.

In a preferred embodiment, control module includes a signal generation unit and a processing unit coupled to the coil structure. The signal generation unit generates and provides to the coil structure an alternating current signal to generate a first electromagnetic signal. The processing unit receives a sensing signal from the coil structure and generates the at least one measurement result of the eddy current induction measurement based on the sensing signal.

In a preferred embodiment, the sensing and stimulation device further includes an isolating element. The isolating element includes a recess to accommodate the coil structure. The coil structure is arranged within the recess with a first side facing away from the ear and towards the isolating element.

Another embodiment according to the present invention is directed to a sensing and stimulation device arranged on an ear of a subject. The sensing and stimulation device includes a sensing module, a stimulation module and a setting structure combined with the sensing module and the stimulation module. The sensing module includes a sensing coil and a sensing controller coupled to the sensing coil. The sensing controller is configured to drive the sensing coil for an eddy current induction measurement to an ear measurement area. The stimulation module includes a stimulation coil and a stimulation controller coupled to the stimulation coil. The stimulation controller is configured to drive the stimulation coil for an electromagnetic stimulation to an ear stimulation area. The setting structure is configured to fix the sensing module and the stimulation module on the ear. The stimulation controller controls at least one parameter of the electromagnetic stimulation based on at least one measurement result of the eddy current induction measurement.

In a preferred embodiment, at least one of the sensing coil and the stimulation coil is arranged on the substrate.

In a preferred embodiment, the ear measurement area corresponds to a vein location of the ear.

In a preferred embodiment, the ear stimulation area corresponds to a parasympathetic nerve location of the ear.

In a preferred embodiment, the at least one measurement result of the eddy current induction measurement includes a heart rate signal of the subject. The heart rate signal includes a low-frequency part and a high-frequency part, and the stimulation controller controls the at least one parameter of the electromagnetic stimulation based on a ratio of the low-frequency part to the high-frequency part.

In a preferred embodiment, the stimulation controller drives the stimulation coil for the electromagnetic stimulation when the ratio is greater than a threshold.

In a preferred embodiment, the sensing controller drives the sensing coil for the eddy current induction measurement after the electromagnetic stimulation, obtains an updated heart rate signal, and determines whether to repeat the electromagnetic stimulation based on the updated heart rate signal.

In a preferred embodiment, sensing controller includes a signal generation unit and a processing unit coupled to the sensing coil. The signal generation unit generates and provides to the sensing coil an alternating current signal to generate a first electromagnetic signal. The processing unit receives a sensing signal from the sensing coil and generates the at least one measurement result of the eddy current induction measurement based on the sensing signal.

In a preferred embodiment, the sensing and stimulation device further includes an isolating element. The isolating element includes a recess to accommodate the sensing coil. The sensing coil is arranged within the recess with a first side facing away from the ear and towards the isolating element.

As described above, the sensing and stimulation device of the present invention includes a setting structure that may be arranged on an ear of a subject, and a module integrating sensing and stimulation. By disposing the sensor on the subject's ear, the measurement influence caused by the subject's action can be reduced. Moreover, disposal on the ear is less likely to affect the subject's action or living than disposal on the limbs or chest. Using a coil to perform eddy current induction measurement on the ear (for example, ear blood vessels or subcutaneous tissue fluid), the subject's physiological signals can be obtained. Compared with optical mechanisms, the eddy current measurement mechanism is not susceptible to physical limitations of skin color or barriers. Compared with blood sampling to measure blood glucose, the eddy current measurement mechanism is non-invasive and will not cause discomfort to the subject. Simultaneous eddy current induction measurement of ear blood vessels or subcutaneous tissue fluid can also obtain blood glucose information and cardiovascular information at the same time, that is favorable to the development of home care or remote care.

### BRIEF DESCRIPTION OF THE DRAWINGS

The drawings presented in the present invention are intended to assist in describing various embodiments of the present invention. However, in order to simplify the drawings and/or highlight the content to be presented in the drawings, conventional structures and/or elements in the drawings may be drawn in a simple schematic manner or may be omitted. On the other hand, the number of elements in the drawings may be singular or plural. The drawings presented in the present invention are for the purpose of illustrating the embodiments only and are not limiting thereof.

FIG. 1 a schematic diagram of a sensing and stimulation device arranged on an ear of a subject in the first embodiment of the present invention.

FIG. 2A is a schematic diagram of the sensing and stimulation device performing the eddy current sensing in the first embodiment of the present invention.

FIG. 2B is a schematic diagram of the sensing and stimulation device performing the electromagnetic stimulation in the first embodiment of the present invention.

FIG. 3 is a signal diagram of at least one measurement result of the sensing and stimulation device in the first embodiment of the present invention.

FIG. 4 is a flowchart illustrating operation of the sensing and stimulation device in the first embodiment of the present invention.

FIG. 5 a schematic diagram of another implementation of the coil structure in the first embodiment of the present invention.

FIG. 6A and FIG. 6B are schematic diagrams of arrangements of the isolating element in the first embodiment of the present invention.

FIG. 7 is a schematic diagram of the operation of the sensing and stimulation device in the second embodiment of the present invention.

FIG. 8 is a circuit block diagram of a sensing module in the second embodiment of the present invention.

FIG. 9 is a circuit block diagram of a stimulation module in the second embodiment of the present invention.

### DETAILED DESCRIPTION

Any reference herein to elements using names such as “first”, “second”, etc. generally does not limit the number or order of these elements. Rather, these names are used herein as a convenient way to distinguish between two or more elements or instances of elements. Therefore, it should be understood that the names “first”, “second”, etc. in the claims do not necessarily correspond to the same names in the written description. Furthermore, it should be understood that reference to first and second elements does not imply that only two elements may be employed or that the first element must precede the second element. The words “comprise”, “include”, “have”, “contain”, etc. used herein are all open terms, which mean including but not limited to.

The term “couple” used herein refers to direct or indirect electrical coupling between two structures. For instance, in an example of indirect electrical coupling, one structure may be coupled to another structure via passive devices like resistors, capacitors or inductors.

In the present invention, the words “exemplary” and “for example” are used to mean “used as an example, instance or illustration”. Any implementation or aspect described herein as “exemplary” or “for example” is not necessarily to be construed as “preferred or advantageous over other aspects of the invention”. The terms “approximately” and “roughly” as used herein with respect to a specified value or characteristic are intended to mean within a certain numerical value (e.g., 10%) of the specified value or characteristic.

The first embodiment according to the present invention is directed to a sensing and stimulation device arranged on an ear of a subject. The sensing and stimulation device includes a coil structure, a setting structure combined with the coil structure, and a control module coupled to coil structure. The setting structure is configured to position the coil structure on the ear. The control module is configured to drive the coil structure for an eddy current induction measurement to an ear measurement area, and to drive the coil structure for an electromagnetic stimulation to an ear stimulation area. The control module controls at least one parameter of the electromagnetic stimulation based on at least one measurement result of the eddy current induction measurement.

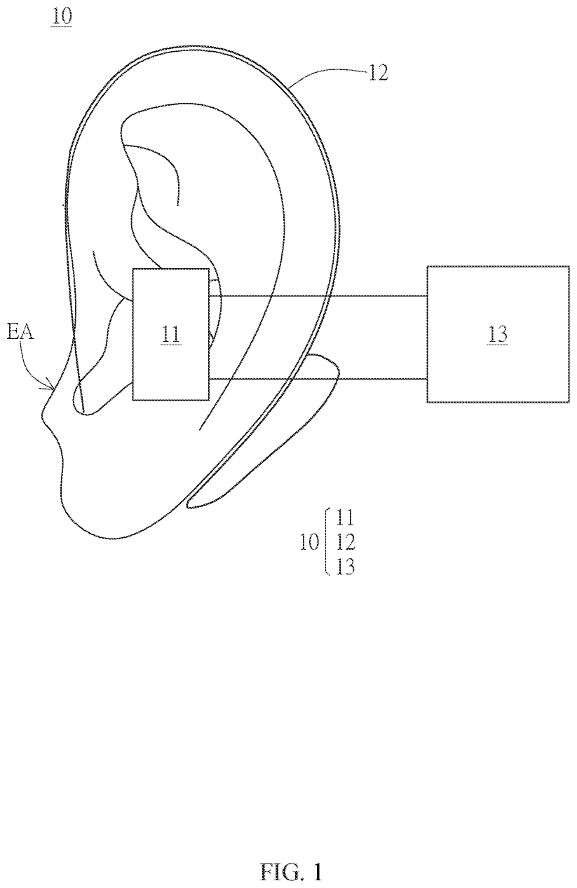

Refer to FIG. 1, which illustrates a sensing and stimulation device 10 arranged on an ear EA of a subject. The sensing and stimulation device 10 includes a coil structure 11, a setting structure 12 combined with the coil structure 11, and a control module 13 coupled to the coil structure 11. The setting structure 12 is configured to position the coil structure 11 on the ear EA.

The coil structure 11 may be positioned on the ear EA of the subject by the setting structure 12. For example, the setting structure 12 may be of hook type, bud type, clip type or piercing type. Specifically, a hook type setting structure may hang or hook on an outer ear part (e.g., helix and/or auricle) of the subject, and the coil structure 11 is positioned on the ear EA through a supporting force provided to the setting structure 12 by the outer ear part. A bud type setting structure 12 has a bud structure like an earphone, positioning the coil structure 11 on the ear EA through a supporting force provided to the setting structure 12 by being plugged into the ear canal entrance and/or leaned against by the tragus of the subject. A clip type setting structure 12 may have a clip structure that may clip locations like an outer ear part (e.g., earlobe and/or auricle) to provide a supporting force for arranging the setting structure 12. A piercing type setting structure 12 may make use of a pierced hole on an outer ear part of the subject, to provide to the setting structure 12 a supporting force needed for positioning the coil structure 11 by a stud structure passing through the pierced hole. It should be noted that the exemplary setting structure 12 is for the purpose of illustrating a setting structure, and not for limiting the present invention. Other setting structures corresponding to any means capable of positioning the coil structure 11 on the ear EA of the subject and placing an emission surface of the coil structure 11 towards an ear measurement area or an ear stimulation area of the subject, such as adhesion, adsorption or tying, should all belong to the scope of the setting structure 12 of the present invention.

It should be noted that although the control module 13 shown in FIG. 1 is not arranged on the ear, the setting structure 12 may also integrate and arrange the control module 13 together in the present embodiment. The present invention does not limit the arrangement location of the control module 13. In highly integrated devices, the control module 13 may be integrated with the setting structure 12 and then arranged on the ear together. In another aspect, if the control module 13 cannot be arranged on the ear, it may be coupled to the coil structure 11 arranged on the ear by means like a transmission line.

The coil structure 11 may be any known inductive device, such as wound with enameled wire into multiple turns or a conductor wiring formed on a substrate. Taking a coil structure on a substrate as an example, known fabrication techniques like etching, engraving, photolithography may be used to form the conductor wiring on the substrate. The coil structure 11 may be of coil pattern like single-turn coil, multi-turn coil or helical coil, but is not limited thereto. Furthermore, the coil structure 11 on the substrate may be planar, for example, coil pattern formed with conductor wiring on a layer of the substrate. In another aspect, the coil structure 11 on the substrate may be stereoscopic, for example, coil pattern formed with conductor wiring in multiple layers of the substrate. The coil count within the coil structure 11 is not limited in the present embodiment; specifically, the coil structure 11 may be at least one coil formed on the substrate. Arranging the coil structure 11 on the substrate may effectively enhance fabrication yield and uniformity of the coil structure 11, and facilitate integration with other circuit devices and modules. If fabricated with a printed circuit board, the coil structure 11 would have further characteristics like low cost, mass production, resistance to deterioration and/or easy cleaning.

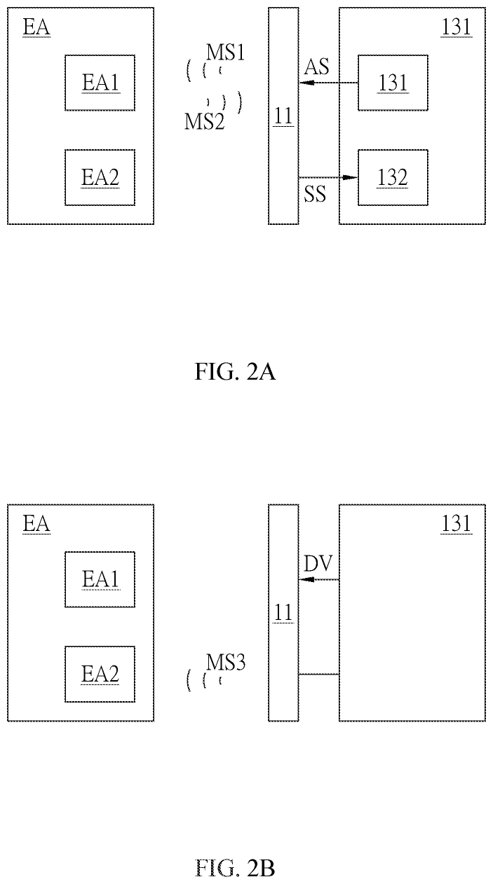

Refer to FIG. 2A and FIG. 2B, which illustrate an implementation of the control module 13 driving the coil structure 11 for an eddy current induction measurement to an ear measurement area EA1, and driving the coil structure 11 for an electromagnetic stimulation to an ear stimulation area EA2. As shown in FIG. 2A, an implementation of eddy current induction measurement is to provide an alternating current signal AS to the coil structure 11 through a signal generation unit 131 of the control module 13, causing the coil structure 11 to emit a first electromagnetic signal MS1 to the ear measurement area EA1. In the ear measurement area EA1, conductive portions (e.g., blood or tissue fluid) inside the ear measurement area EA1 can be regarded as a planar conductor. The planar conductor would generate an eddy current induced by the first electromagnetic signal MS1, and generate a corresponding second electromagnetic signal MS2. The coil structure 11 would generate a sensing signal SS due to the mutual influence of the first electromagnetic signal MS1 and the second electromagnetic signal MS2. Based on the sensing signal SS, a processing unit 132 of the control module 13 can obtain at least one measurement result of the eddy current induction measurement. It should be noted that the sensing signal SS may be based on impedance variation, magnetic flux variation or other values of the coil structure 11 useful in evaluating the second electromagnetic signal MS2. The distribution variation, volume variation and/or component variation of conductive portions inside the ear measurement area EA1 may be determined through the sensing signal SS. Taking blood in the ear measurement area EA1 as an example, the ear measurement area EA1 preferably corresponds to a vein location of the ear, especially the postauricular vein. Veins contract or relax due to pulse, and the blood in the veins also varies in volume or cross-sectional area due to contraction or relaxation. The sensing signal SS would also vary due to the pulse, so the at least one measurement result of the eddy current induction measurement may correspond to a heart rate signal of the subject. However, the at least one measurement result of the eddy current induction measurement is not limited to the heart rate signal of the subject in the present invention.

In another aspect, refer to FIG. 2B, which illustrates an implementation of the control module 13 driving the coil structure 11 for an electromagnetic stimulation to the ear stimulation area EA2. After completing the eddy current induction measurement and obtaining at least one measurement result, the control module 13 may regulate at least one parameter of the electromagnetic stimulation based on the at least one measurement result. The at least one parameter of the electromagnetic stimulation includes, for example, frequency, magnetic flux, magnetic induction intensity, duration, and/or on/off. The electromagnetic stimulation may provide a driving voltage DV to the coil structure 11 through the control module 13. The coil structure 11 generates a stimulation electromagnetic signal MS3 due to being driven by the driving voltage DV, and provides it to the ear stimulation area EA2. The ear stimulation area EA2 is, for example, a densely packed area of the ear nerve plexus, and is preferably a parasympathetic nerve location (e.g., within the ear canal) of the ear. By using electromagnetic stimulation on the parasympathetic nerves in the ear, the stimulation of the sympathetic and parasympathetic nerves is balanced. It should be noted that the driving voltage DV can be provided through the signal generation unit 131 or provided by an independent component.

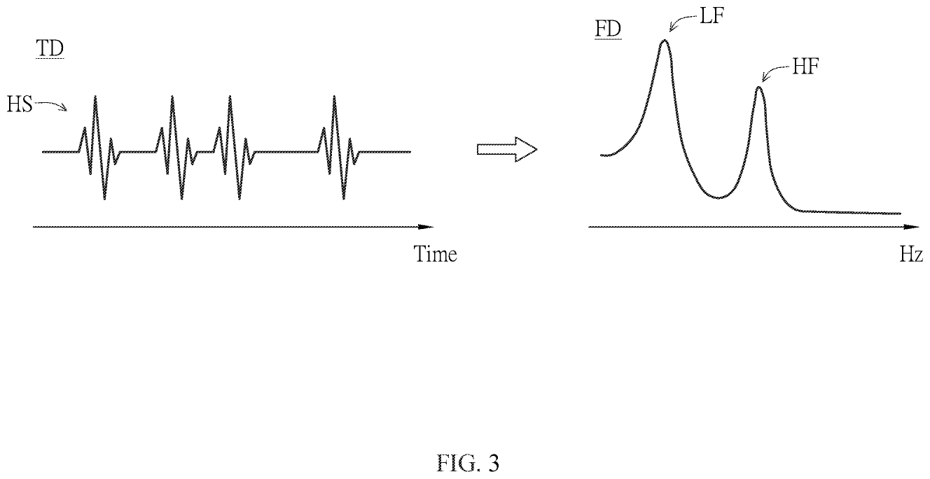

Refer to FIG. 3, which illustrates a correlation between at least one measurement result of the eddy current induction measurement and sympathetic/parasympathetic nerves. By transforming the subject's heart rate signal from the time domain TD to the frequency domain FD, a high-frequency part HF and a low-frequency part LF can be obtained. The high-frequency part HF reflects the function of parasympathetic nerves. The ratio of the low-frequency part LF to the high-frequency part HF (LF/HF) can be regarded as an indicator of sympathetic nerve function. More specifically, methods such as Fourier transformation or autoregressive models can be used to transform the heart rate signal HS from the time domain TD to the frequency domain FD. Based on the frequency domain FD analysis results, the power density in different frequency ranges in the heart rate signal HS can be calculated to obtain an autonomic nervous system balance index. Autonomic nervous system balance indicators include the low-frequency part LF (0.04 Hz to 0.15 Hz) which represents sympathetic nerve activity, and the high-frequency part HF (0.15 Hz to 0.4 Hz) which represents parasympathetic nerve activity. After calculating the corresponding power values of the low-frequency part LF and the high-frequency part HF, the autonomic nervous system balance index can be calculated, that is, the ratio of the power of the low-frequency part to the power of the high-frequency part (LF/HF). If the ratio (LF/HF) is greater than 1, the sympathetic nerve activity is relatively higher, whereas if the ratio (LF/HF) is less than 1, the parasympathetic nerve activity is relatively higher. Relatively higher sympathetic activity is typically associated with stress, irritation, or increased sympathetic activity, whereas relatively higher parasympathetic activity is typically associated with relaxation, rest, or increased parasympathetic activity.

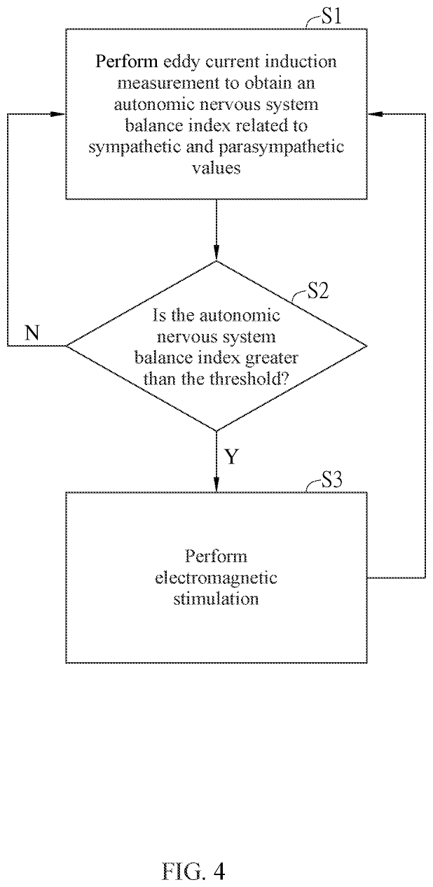

Refer to FIG. 4 for a flowchart illustrating operation of the sensing and stimulation device 10. In step S1, eddy current induction measurement is performed to obtain an autonomic nervous system balance index related to sympathetic and parasympathetic values. In step S2, the autonomic nervous system balance index is judged. If the autonomic nervous system balance index is less than a threshold (e.g., the ratio (LF/HF) is less than 1) in step S2, eddy current induction measurement is continued to continuously monitor the subject's autonomic nervous system balance index; if the autonomic nervous system balance index is greater than the threshold, step S3 is entered. In step S3, electromagnetic stimulation is performed; the control module 13 adjusts various parameters of the electromagnetic stimulation based on the results of the eddy current induction measurement. For example, if the sympathetic nerve activity is too strong (the ratio (LF/HF) is much greater than 1), the frequency or intensity of stimulation can be increased to achieve a rapid balancing effect, but is not limited hereto. After step S3, the process will return to step S1 to continue to perform eddy current induction measurement and continuously monitor the subject's autonomic nervous system balance index. It should be noted that the threshold in the present invention is not limited to be only ‘1’, but may be adjusted according to clinical data.

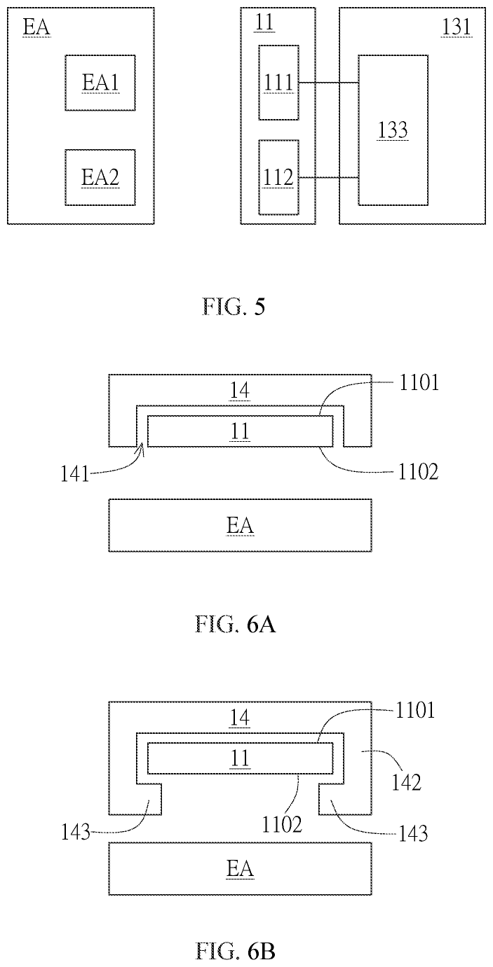

In one implementation, the coil structure 11 may be two standalone coil units. For example, refer to FIG. 5, which illustrates a coil structure 11 including a first coil unit 111 and a second coil unit 112. The first coil unit 111 is arranged corresponding to the ear measurement area EA1. The second coil unit 112 is arranged corresponding to the ear stimulation area EA2. The control module 13 may include a switch 133 to couple switchably to the first coil unit 111 or the second coil unit 112. Through standalone coil units, the arrangement of the coil structure 11 is not limited to the ear measurement area EA1 and the ear stimulation area EA2. When the ear measurement area EA1 and the ear stimulation area EA2 are too separated, using standalone coil units for measurement can overcome spatial obstacles. Moreover, by performing the eddy current measurement and the electromagnetic stimulation respectively on the first coil unit 111 and the second coil unit 112, the thermal effect caused by electromagnetic conversion of the first coil unit 111 and the second coil unit 112 can be reduced, thereby avoiding coil overheating or measurement inaccuracy and other issues.

In one implementation, external interference to the coil structure 11 can be reduced through an isolating element. Refer to FIG. 6A, which illustrates the sensing and stimulation device 10 further including an isolating element 14. The isolating element 14 includes a recess 141 to accommodate the coil structure 11. The coil structure 11 is arranged within the recess 141 with a first side 1101 facing away from the ear and towards the isolating element 14, specifically, to place an emission surface for the first electromagnetic signal MS1 or the stimulation electromagnetic signal MS3 of the coil structure 11 (i.e., second side 1102) primarily towards the ear measurement area EA1 or the ear stimulation area EA2. Shielding materials or shielding circuits can be applied outside the first side 1101 of the coil structure 11 to reduce influences from outside the first side 1101, such as interference in wirings and electromagnetic wave interference in the environment. In FIG. 6B, a recess wall 142 of the recess 141 of the isolating element 14 may have a limiting structure 143, which can further make the first electromagnetic signal MS1 or the stimulation electromagnetic signal MS3 more directive, and keep the first electromagnetic signal MS1 or the stimulation electromagnetic signal MS3 from affecting the non-measurement or stimulation area. Using a more directive first electromagnetic signal MS1 or stimulation electromagnetic signal MS3 can further reduce the output power of the control module 13 and alleviate the possible harm of electromagnetic waves.

The sensing and stimulation device of the present invention includes a setting structure that may be arranged on an ear of a subject, and a module integrating sensing and stimulation. By the setting structure, the impact of the sensing and stimulation device on the subject's actions can be reduced, and the impact of the subject's actions on the accuracy of sensing and/or stimulation can also be reduced. In another aspect, the module integrating sensing and stimulation can adjust stimulation parameters through sensing feedback. Finally, the present invention uses electrode-free sensing or stimulation mechanisms such as electromagnetic stimulation and eddy current sensing, which can avoid the aforementioned shortcomings of traditional contact electrodes.

The second embodiment according to the present invention is directed to a sensing and stimulation device arranged on an ear of a subject. The sensing and stimulation device includes a sensing module, a stimulation module and a setting structure combined with the sensing module and the stimulation module. The sensing module includes a sensing coil and a sensing controller coupled to the sensing coil, wherein the sensing controller is configured to drive the sensing coil for an eddy current induction measurement to an ear measurement area. The stimulation module includes a stimulation coil and a stimulation controller coupled to the stimulation coil, wherein the stimulation controller is configured to drive the stimulation coil for an electromagnetic stimulation to an ear stimulation area. The setting structure is configured to position the sensing module and the stimulation module on the ear. The stimulation controller controls at least one parameter of the electromagnetic stimulation based on at least one measurement result of the eddy current induction measurement.

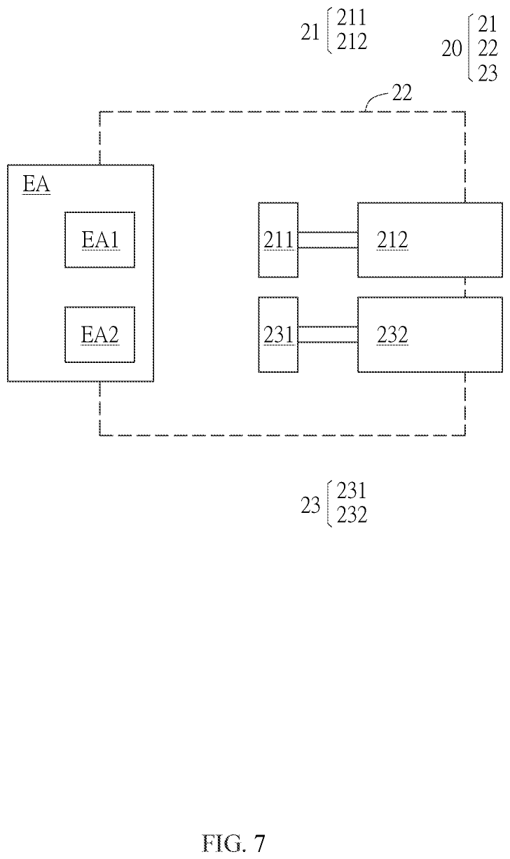

Refer to FIG. 7, which illustrates a sensing and stimulation device 20 arranged on an ear of a subject. The sensing and stimulation device 20 includes a sensing module 21, a setting structure 22 and a stimulation module 23. The sensing module 21 includes a sensing coil 211 and a sensing controller 212 coupled to the sensing coil 211. The stimulation module 23 includes a stimulation coil 231 and a stimulation controller 232 coupled to the stimulation coil 231.

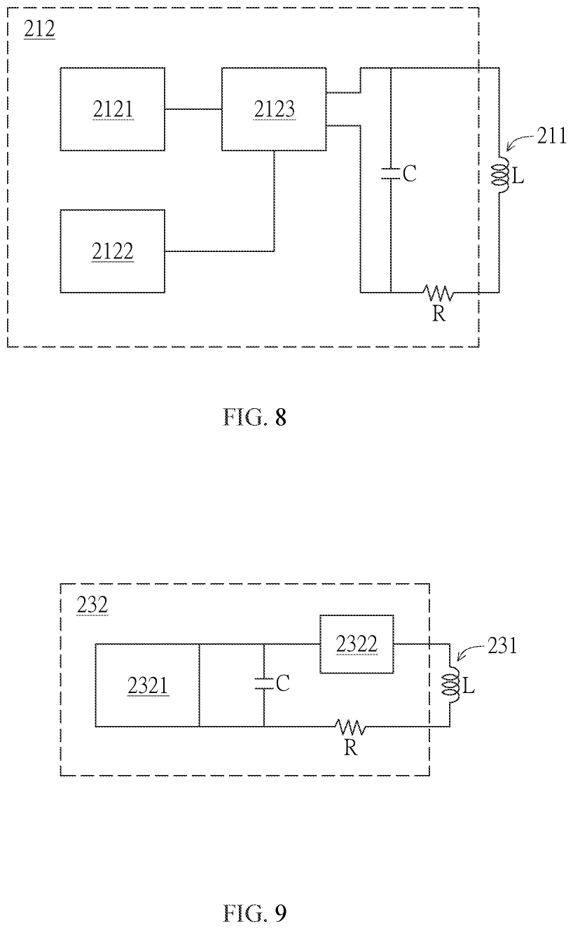

In one implementation of the sensing module, refer to FIG. 8 for a circuit block diagram of the sensing module 21. The sensing coil 211 (equivalent to an inductor L) is coupled to a series circuit of a resistor R and a capacitor C. Selected through the switching unit 2123 in the sensing controller 212, the sensing coil 211 is coupled to a signal generation unit 2121 or a processing unit 2122. Since the sensing coil 211 is coupled to the series circuit of the resistor R and the capacitor C, the frequency of the electromagnetic signal emitted by the sensing coil 211 can be adjusted by adjusting the impedance value of the series circuit. However, the method of adjusting the frequency of the electromagnetic signal is not limited hereto. The signal generation unit 2121 may generate different alternating current signals to achieve the purpose of adjusting the frequency of the electromagnetic signal. The frequency of the electromagnetic signal may be adjusted for samples of different depths or required resolution, but is not limited hereto.

In one implementation of the stimulation module, refer to FIG. 9 for a circuit block diagram of the stimulation module 23. The stimulation controller 232 may include a voltage source 2321, a capacitor C coupled to the stimulation coil 231, and a discharge switch 2322. Specifically, in order to efficiently operate the transmembrane potential of neurons, a current of high time-varying rate needs to be passed into the stimulation coil 231 to generate sufficient electric field stimulation in the ear stimulation area. The current variation in the stimulation coil 231 is determined by the charging voltage of the capacitor C and the inductance value of the stimulation coil 231. In order to achieve the electric field induced by high current pulses, for example, means such as increasing the capacitance value or voltage value of the capacitor C, or reducing the inductance value of the stimulation coil 231 may be adopted. It should be noted that the circuit architectures of the sensing module 21 and the stimulation module 23 depicted in FIG. 8 to FIG. 9 are only for illustration and do not limit the present invention.

In the present embodiment, an isolating element may be arranged to accommodate the sensing coil or the stimulation coil. For example, the isolating element may include a recess to accommodate the sensing coil or the stimulation coil. The sensing coil or the stimulation coil is arranged within the recess with a side facing away from the ear and towards the isolating element. It should be noted that the isolating element used in the stimulation coil can prevent the stimulation signal of the stimulation coil from affecting the back-end circuit or the non-ear stimulation area, but it is not limited hereto.

Compared with the first embodiment, the present embodiment can reduce the size of the control module by applying standalone sensing and stimulation modules to perform the eddy current induction measurement and the electromagnetic stimulation, respectively. When the ear space cannot accommodate a larger module, the control module can be divided into two small modules. And by arranging the setting structure on the ear, the comfort and convenience of wearing can be improved.

The preceding description of the present invention is provided so that a person of ordinary skill in the art can produce or implement the present invention. For a person of ordinary skill in the art, various modifications to the present invention will be obvious, and without departing from the spirit or scope of the present invention, the general principles defined herein can be applied to other variants, or the embodiments may be combined with each other or implemented separately. Therefore, the present invention is not intended to be limited to the examples described herein, but should be accorded the broadest scope of the principles and novel features disclosed herein.

What is claimed is:

1. A sensing and stimulation device arranged on an ear of a subject, comprising:
a coil structure;
a setting structure combined with the coil structure, wherein the setting structure is configured to position the coil structure on the ear; and
a control module coupled to the coil structure, wherein the control module is configured to drive the coil structure for an eddy current induction measurement to an ear measurement area, and to drive the coil structure for an electromagnetic stimulation to an ear stimulation area,
wherein the control module controls at least one parameter of the electromagnetic stimulation based on at least one measurement result of the eddy current induction measurement.

2. The sensing and stimulation device of claim 1, wherein the coil structure includes:
a first coil unit arranged corresponding to the ear measurement area; and
a second coil unit arranged corresponding to the ear stimulation area.

3. The sensing and stimulation device of claim 1, wherein the coil structure includes:
a substrate; and
at least one coil arranged on the substrate.

4. The sensing and stimulation device of claim 1, wherein the ear measurement area corresponds to a vein location of the ear.

5. The sensing and stimulation device of claim 1, wherein the ear stimulation area corresponds to a parasympathetic nerve location of the ear.

6. The sensing and stimulation device of claim 1, wherein the at least one measurement result of the eddy current induction measurement includes a heart rate signal of the subject;
wherein the heart rate signal includes a low-frequency part and a high-frequency part, and the control module controls the at least one parameter of the electromagnetic stimulation based on a ratio of the low-frequency part to the high-frequency part.

7. The sensing and stimulation device of claim 6, wherein the control module drives the coil structure for the electromagnetic stimulation when the ratio is greater than a threshold.

8. The sensing and stimulation device of claim 7, wherein the control module drives the coil structure for the eddy current induction measurement after the electromagnetic stimulation, obtains an updated heart rate signal, and determines whether to repeat the electromagnetic stimulation based on the updated heart rate signal.

9. The sensing and stimulation device of claim 1, wherein the control module includes:
a signal generation unit coupled to the coil structure for generating and providing to the coil structure an alternating current signal to generate a first electromagnetic signal; and
a processing unit coupled to the coil structure for receiving a sensing signal from the coil structure and generating the at least one measurement result of the eddy current induction measurement based on the sensing signal.

10. The sensing and stimulation device of claim 1, further comprising:
an isolating element including a recess to accommodate the coil structure, wherein the coil structure is arranged within the recess with a first side facing away from the ear and towards the isolating element.

11. A sensing and stimulation device arranged on an ear of a subject, comprising:
a sensing module including:
a sensing coil; and
a sensing controller coupled to the sensing coil, wherein the sensing controller is configured to drive the sensing coil for an eddy current induction measurement to an ear measurement area;
a stimulation module including:
a stimulation coil; and
a stimulation controller coupled to the stimulation coil, wherein the stimulation controller is configured to drive the stimulation coil for an electromagnetic stimulation to an ear stimulation area; and
a setting structure combined with the sensing module and the stimulation module, wherein the setting structure is configured to position the sensing module and the stimulation module on the ear;
wherein the stimulation controller controls at least one parameter of the electromagnetic stimulation based on at least one measurement result of the eddy current induction measurement.

12. The sensing and stimulation device of claim 11, wherein at least one of the sensing coil and the stimulation coil is arranged on a substrate.

13. The sensing and stimulation device of claim 11, wherein the ear measurement area corresponds to a vein location of the ear.

14. The sensing and stimulation device of claim 11, wherein the ear stimulation area corresponds to a parasympathetic nerve location of the ear.

15. The sensing and stimulation device of claim 11, wherein the at least one measurement result of the eddy current induction measurement includes a heart rate signal of the subject;
wherein the heart rate signal includes a low-frequency part and a high-frequency part, and the stimulation controller controls the at least one parameter of the electromagnetic stimulation based on a ratio of the low-frequency part to the high-frequency part.

16. The sensing and stimulation device of claim 15, wherein the stimulation controller drives the stimulation coil for the electromagnetic stimulation when the ratio is greater than a threshold.

17. The sensing and stimulation device of claim 16, wherein the sensing controller drives the sensing coil for the eddy current induction measurement after the electromagnetic stimulation, obtains an updated heart rate signal, and determines whether to repeat the electromagnetic stimulation based on the updated heart rate signal.

18. The sensing and stimulation device of claim 11, wherein the sensing controller includes:
a signal generation unit coupled to the sensing coil for generating and providing to the sensing coil an alternating current signal to generate a first electromagnetic signal; and
a processing unit coupled to the sensing coil for receiving a sensing signal from the sensing coil and generating the at least one measurement result of the eddy current induction measurement based on the sensing signal.

19. The sensing and stimulation device of claim 11, further comprising:
an isolating element including a recess to accommodate the sensing coil or the stimulation coil, wherein the sensing coil or the stimulation coil is arranged within the recess with a first side facing away from the ear and towards the isolating element.
